(19) United States
(12) Patent Application Publication
(10) Pub. No.: US 2022/0400968 A1
(43) Pub. Date: Dec. 22, 2022

(54) SYSTEM AND METHOD FOR MEASURING INTRACRANIAL PRESSURE

(71) Applicants: Sheryl Rae Thingvold, Toronto (CA); Sebastian Flaminius Marius Koga, Covington, LA (US); Wesley Bryan Hodges, London (CA)

(72) Inventors: Sheryl Rae Thingvold, Toronto (CA); Sebastian Flaminius Marius Koga, Covington, LA (US); Wesley Bryan Hodges, London (CA)

(21) Appl. No.: 17/842,631

(22) Filed: Jun. 16, 2022

Related U.S. Application Data
(60) Provisional application No. 63/211,803, filed on Jun. 17, 2021.

(51) Int. Cl. A61B 5/03; A61B 8/08; A61B 5/00

(52) U.S. Cl. CPC A61B 5/031; A61B 8/0808; A61B 5/6817; A61B 8/5223; A61B 2562/0247

(57) ABSTRACT

Embodiments of the present invention relate to a system and method for measuring intracranial pressure (ICP). Embodiments of the present invention include emitting an electromagnetic wave into the temple area and/or inner ear of a patient and measuring ICP based on the characteristics of the reflected and/or transmitted electromagnetic wave scattered by the tissue and/or cavity. The characteristics may include variations in the electromagnetic wave corresponding to distortions by the cavity within the skull beneath the temple and/or the oval window within the patient's inner ear. Further, embodiments of the present invention include ICP is elevated in the patient. The present invention concentrates on measuring and quantifying the changes in transmission characteristics to determine changes in ICP.

The instant patent application claims priority to U.S. Provisional application Ser. No. 63/211,803, filed on Jun. 17, 2021, presently pending.

### TECHNICAL FIELD

The present disclosure relates generally to a system and method for measuring intracranial pressure (ICP) and more specifically to an electromagnetic system for non-invasive measurement of ICP and a method for using the electromagnetic system to measure ICT.

### BACKGROUND

This section is intended to introduce the reader to various aspects of art that may be related to various aspects of the present invention. This discussion is believed to be helpful in providing the reader with background information to facilitate a better understanding of the various aspects of the present invention. Accordingly, it should be understood that these statements are to be read in this light, and not as admissions of prior art.

The human skull is essentially a rigid fluid-filled container. Principal constituents within the skull include brain tissue, blood, and cerebral-spinal fluid (CSF). Because the skull is essentially rigid and has a constant volume, if there is an increase in the volume of the contents of the skull, the pressure inside the skull (i.e., intracranial pressure. ICP) will rise unless some fluid is able to escape. For example, if the brain tissue experiences swelling, a certain amount of blood or CSF must escape the skull cavity to prevent a rapid increase in pressure. During such swelling, pressure inside the skull may rise above the normal range. Further, if swelling continues until little or no fluid remains, any further swelling will cause a rapid increase in ICP.

ICP is measured in millimeters of mercury (mmHg). The normal range for ICP values is from around 5 mmHg to around 13 mmHg. American and European head injury guidelines recommend that actions be taken to treat ICP when it is above 20-25 mmHg, as elevated ICP is a potentially life-threatening condition. Treatment of elevated ICP typically begins with administration of drugs to reduce systemic fluid volume or blood pressure. If the elevated ICP is not detected early enough, part of the skull may need to be removed to relieve the pressure.

While elevated ICP is often a result of trauma, the elevated pressure itself can cause damage to the central nervous system by compressing important brain structures and restricting blood flow through vessels that supply the brain. Elevated ICP typically occurs as a result of increased volume within the skull cavity. For example, elevated ICP occurs acutely in head trauma cases involving cerebral edema, which is also referred to as brain swelling. Elevated ICP may occur more gradually in cases of hydroencephalitis (i.e., water on the brain) or brain tumors. Other conditions that may cause elevated ICP include: subdural hematoma, encephalitis, meningitis, hemorrhage, stroke, and so forth.

Traditional techniques for monitoring and measuring ICT generally involve the use of invasive devices. For example, commonly used devices include hollow screw and bolt devices. These typically include metallic cylindrical instruments which are inserted into the patient such that an instrument tip protrudes into the subarachnoid space to facilitate pressure measurement. The subarachnoid space is the compartment within the skull and spinal column that contains the CSF. Another commonly used invasive device for ICP monitoring is an intraventricular catheter. The intraventricular catheter is typically placed inside ventricles (i.e., fluid-filled cavities) of the brain to facilitate pressure monitoring. Insertion of such invasive devices (e.g., hollow screws and catheters) to facilitate ICP monitoring can be dangerous. For example, insertion of a monitoring device through a patient's skull may cause hemorrhaging or infection. In many different medical settings, it would be advantageous to be able to monitor changes in bodily fluids as they occurred in a non-invasive manner. For example, it is often critical to measure intracranial changes in fluid in an intensive care unit patient. Standard of care for these patients includes invasive monitors that require drilling a hole in the cranium and inserting a probe such as an ICP monitor, or microdialysis or “licox”probes for measuring chemical changes to the fluids in the brain. Non-invasive measurement techniques are not currently commercially available for detecting cerebral fluid changes such as would occur with bleeding or edema, and many brain injuries are not severe enough to warrant drilling a hole in the cranium for invasive monitoring. Thus. for many patients with brain injury, there is no continuous monitoring technology available to alert clinical staff when there is a potentially harmful increase in edema. or bleeding. instead, these patients are typically observed by nursing staff, employing a clinical neurological examination, and it is not until increased fluid in the brain causes observable brain function impairment that the physicians or nurses can react. In other words, there is no way currently available for monitoring intracranial fluid changes themselves, and thus the ability to compensate for such changes is limited.

### SUMMARY

In the present disclosure, there is described a system and method for non-invasive direct measurement of ICP.

In a first aspect of the present invention, there is provided an ICP measurement system comprising a sensor, the sensor further comprising a transmitter for transmitting an electromagnetic signal through a skull bone to an intracranial area and a receiver for receiving a. reflected electromagnetic signal, a central processor unit to receive the reflected electromagnetic signal from the receiver and reconstruct the reflected electromagnetic signal into an ICP wave, and a processor to analyze the ICP wave and determine an ICP pressure.

In a further aspect of the present invention, there is provided a method for measuring an ICP, the method comprising placing a sensor in proximity to a patient head structure, the sensor further comprising a transmitter and a receiver, transmitting an electromagnetic signal from the sensor through a skull bone to an intracranial area, receiving a reflected electromagnetic signal to the sensor, transmitting the reflected electromagnetic signal from the sensor to a central processing unit, reconstructing the reflected electromagnetic signal to obtain an ICP wave, and analyzing the ICP wave to determine an ICP.

A more complete understanding of the ICP measurement system and method can be obtained by reference to the following detailed description in connection with the attached drawings.

### BRIEF DESCRIPTION OF THE DRAWINGS

FIG. 1A is a perspective view and a block diagram of a system for monitoring a patient's ICP via the patient's temple area in accordance with an exemplary embodiment of the present invention.

FIG. 1B is an exploded view of the embodiment of the sensor shown in FIG. 1A.

FIG. 2A is a frontal view of a further exemplary embodiment of the present invention for monitoring a patient's ICP via the patient's ear canal.

FIG. 2B is a schematic and cutaway view of the embodiment of the present invention provided in FIG. 2A.

FIG. 3 is a schematic view of the temporal area/temporal bone anatomy and the cranial spaces.

FIG. 4 is a schematic view of the inner ear and the cranial spaces illustrating the communication between the cranial spaces and the oval window.

The drawings presented herein are presented for convenience to explain the functions of the elements included in the described embodiments of the system. Elements and details that are obvious to the person skilled in the art may not have been illustrated. Conceptual sketches have been used to illustrate elements that would be readily understood in the light of the present disclosure. Some details have been exaggerated for clarity. These drawings are not fabrication drawings and should not be scaled.

### DETAILED DESCRIPTION

The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure. It should be appreciated that in the development of any such actual implementation, as in any engineering or design project, numerous implementation-specific decisions may be made to achieve the developers' specific goals, such as compliance with system-related and business-related constraints, which may vary from one implementation to another. Moreover, it should be appreciated that such a development effort might be complex and time-consuming, but would nevertheless be a routine undertaking of design, fabrication, and manufacture for those of ordinary skill having the benefit of this disclosure.

Embodiments of the present invention relate to using scattered electromagnetic signals obtained from the temple or inner ear of a patient's skull to non-invasively detect and/or measure intracranial pressure (ICP). For example, in some embodiments, a. sensor including an antenna may be applied to a patient's temple area to transmit an electromagnetic wave within the skull and cavity beneath and collect and/or analyze the scattered signal to determine the patient's ICP. In other embodiments, the sensor may be inserted within the patient's ear canal to transmit an electromagnetic wave directed towards the cavity containing the oval window and collect and/or analyze the scattered signal to determine the patient's ICP. Accordingly, embodiments of the present invention include collecting and/or analyzing a reflected electromagnetic signal to identify and/or quantify ICP.

The electromagnetic signal is transmitted by an antenna located in proximity to the patient's skull, for example, in the temple area or within the ear canal. The antenna. may comprise a microstrip antenna, slotline antenna, or printed antenna, composed of one or more elements. The transmitter and receiver may also comprise two different antennas.

In addition to an antenna, the sensor also may include a matching layer, for matching the impedance of the antenna to the transmission medium.

A substrate backs the conductive portion of the antenna, wherein the selection of the material of the substrate influences the antenna's properties.

The signal distortion of the reflected signal is analyzed to determine the patient's ICP. Properties such as resonant frequency, signal amplitude, phase shifts, polarization, and wavelength may be evaluated. The electromagnetic signal may be radio wave, infrared signal, or microwave.

The system may include a narrowband microwave transceiver and signal processing to determine the ICP from the reflected electromagnetic signal.

An artificial intelligence supportive platform may also be used for diagnosis as part of the analysis of the reflected signal.

The ICP measurement system may be used to measure ICP at a point in time as an “instant-read” capacity, or may be used to continuously monitor a patient ICP.

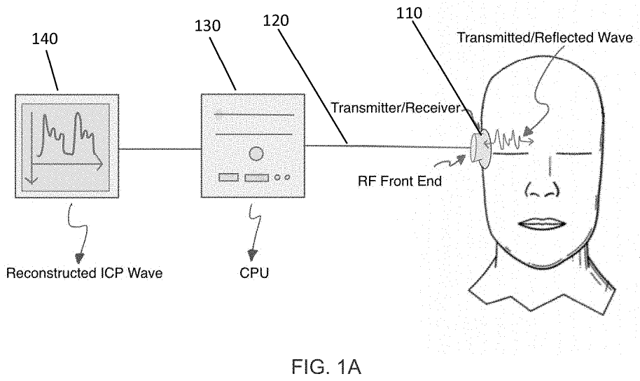

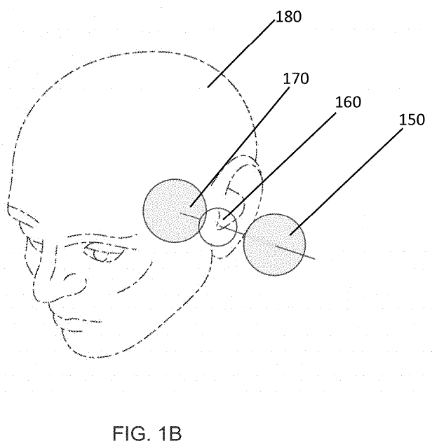

Referring to FIG. 1A, a system for measuring ICP, is illustrated in accordance with an exemplary embodiment of the present invention. A temple sensor 110 may be coupled to a cord 120 to enable communication between components of the sensor (e.g., the measurement device, not shown), a signal processor 130, and a monitor 140. The cord 120 may also supply power to the sensor 110. In other embodiments, the sensor 110 may be powered by a battery and communicate wirelessly with the signal processor 130 and the monitor 140.

As further illustrated in FIG. 1B, the sensor 110 includes a cover 150, an insulating and/or cushioning layer (not shown), an electromagnetic transmission and receiving device 160, a matching layer 170, and an adhesive layer (not shown). The cover 150 serves as a protective outer layer for the sensor 110 and is exposed to the environment when the sensor is attached to a patient's skin. The cover 150 may be made of polyvinyl chloride (PVC) foam, urethane foam material, or the like. The cover 150 at least partially covers and protects the electromagnetic measurement device 160, which includes an emitter and a detector. In one embodiment, the emitter is configured to generate an electromagnetic wave and is configured to detect the scattered signal. The sensor is configured such that an electromagnetic signal from the emitter can be directed at a patient's skin in the temple area of the skull and scattered through the patient's tissue. The electromagnetic signal that is reflected by the patient's tissue and underlying cavity will vary in accordance with the patient's intracranial pressure. Accordingly, the amount of signal distortion detected by the detector can be utilized to measure intracranial pressure. In another embodiment, the emitter and detector may be housed separately, with the emitter situated on the patient's temple and the detector sitting elsewhere on the patient's skull, for example, on the opposing temple.

The velocity of the transit of electromagnetic waves in a vacuum is equal to the inverse of the square root of the product of magnetic permeability and electric permittivity. This formula yields the well-known value of the speed of light of approximately 3×10⁸ meters/second. The finite time required for an electromagnetic field to propagate through a medium, or be reflected by that medium, however, results in a time delay, which is manifested as a phase shift (e.g., an offset or a delay) between a field emitted from a transmitter as compared with the field as sensed at a receiver. In other words, electromagnetic fields typically propagate fastest in a vacuum and propagate slower if any matter or medium is present between the transmitter and the receiver. The amount of slowing is inversely proportional to the square root of the product of the relative permeability and relative permittivity of the medium.

The material makeup of biological materials is almost entirely non-magnetic, with a relative permeability of approximately 1. The variation in the time delay/phase shift through biological materials may therefore be mainly dependent on the average relative permittivity along the path through which the electromagnetic field passes. Relative permittivity varies for various tissue types and body fluids. The permittivity of the biological materials may also depend on the frequency of a time-varying electromagnetic field and may depend on the ambient temperature. The relative permittivity of body fluids is higher than most brain and surrounding tissues, and thus, changes in fluid levels in the brain may have a relatively large effect on the overall phase shift of electromagnetic fields as they propagate through a brain and/or inner ear region, or other medium.

For radiofrequency (“RF”) frequencies below about 200 MHz, the distance between opposing sides of the brain is less than one wavelength for normally propagating transverse electromagnetic waves. This is known as the near field, and in this region, the electromagnetic waves are not fully formed. For this near-field magnetic field propagation case, the propagation time and phase change is predominantly determined by the loss factor of the tissues and liquids in the path rather than their relative permittivity. The loss factor is a function of the imaginary portion of the complex permittivity and the conductivity. The physical mechanism for dissipation of energy is the constant realignment of polarized molecules to the changing field polarity. Therefore the loss factor for a given substance is largely dependent on its ionic content. The ionic content of the brain and surrounding tissues and brain and inner ear liquids is different for each substance. When combined with variations in relative permittivity, the various biological tissues and liquids in the brain and surrounding tissue display unique phase signatures when looking at phase changes for both the lower frequency near field propagation and higher frequency normal propagation cases. Because of the major difference in the physics that causes the phase delay, a multi-spectral measurement using RF frequencies both below and above 200 MHz allows characterization of not only the fractional amount of liquid in the brain, but sub-classifications of the exact nature of the liquid content such as the fractions of blood, cerebrospinal fluid (CSF), or the other liquids that accumulate in the cerebral cavity due to hemorrhaging or edema.

The adhesive layer is disposed on the matching layer 170, on the outer portion of the sensor opposite the cover 150, and is adapted to facilitate attachment of the sensor 110 to a patient 180. In the illustrated embodiment of FIG. 1B, the adhesive layer is essentially circularly shaped and adheres the sensor 110 (antenna) to the patient's temple area. Further, the adhesive layer may include a thermally stable adhesive material to avoid compromised performance when the sensor 110 is exposed to heat. In one embodiment, the adhesive layer includes a plastic strip having acrylic adhesive on one side for attachment to the patient. In another embodiment, the adhesive layer includes multiple adhesive sheets.

The system may be used to measure ICP at a point in time as an “instant-read” capacity, or may be coupled to a patient 180 to allow continuous monitoring of the patient's ICP. Specifically, the system is coupled to the patient via the sensor 110, which is attached to the patient's temple area and held in place by adhesive.

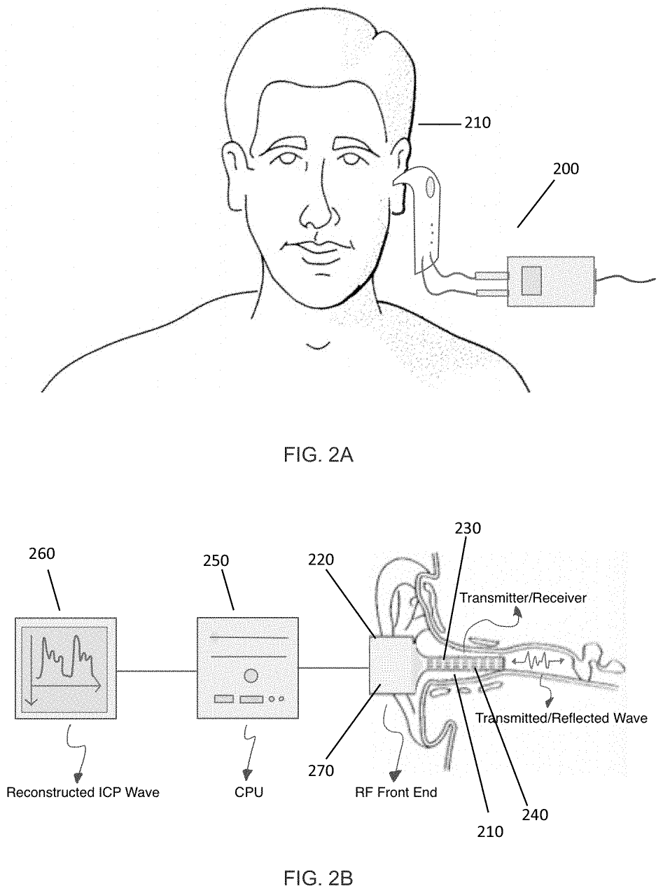

FIG. 2 illustrates a further embodiment of the system for non-invasively monitoring a patient's ICP, in which the sensor is positioned within a patient's ear canal.

FIG. 2A provides a frontal view of the ICP monitoring system 200 placed in an ear canal of a patient 210.

In FIG. 2B, a schematic view of the ICP monitoring system 200 with a cutaway view of the ear canal 210 is provided. The system includes a sensor 220 with an extended end 230 to fit into the ear canal 210, a casing 240 which positions the sensor 220 within the ear canal, a CPU 250, and a monitor 260. The monitor 260 may include a vital signs monitor (e.g., a pulse rate monitor, a pulse oximeter) and/or a pressure mapping device. For example, the monitor 260 may be adapted to receive input from the transceiver of the sensor 220 relating to measuring ICP.

A cover 270 serves as a protective outer layer for the sensor 220 and may be made of polyvinyl chloride (PVC) foam, urethane foam material, or the like. The cover 270 protects the electromagnetic measurement device of the sensor 220, which includes an emitter and a detector. In one embodiment, the emitter is configured to generate an electromagnetic wave and is configured to detect the scattered signal. The electromagnetic signal that is reflected by the oval window of the inner ear and surrounding cavity/tissue will vary in accordance with the patient's intracranial pressure. Accordingly, the amount of signal distortion detected by the detector can be utilized to measure intracranial pressure. In another embodiment, the emitter and detector may be housed separately, with the emitter situated within the patient's inner ear canal and the detector sitting elsewhere on the patient's skull, for example within the opposing inner ear canal.

The system is positioned within the patient's ear canal 210 to allow for monitoring of the patient's ICP. The system may require calibration, specifically, steering the transceiver electrically to scatter the patient's oval window with a signal that is captured and analyzed to determine ICP. This calibration may be accomplished by rotating the beam through a 180-degree endfire cycle and interrogating the tissue until the location of the oval window is identified. The transceiver will then be trained on this location to ultimately measure the patient's ICP in real-time.

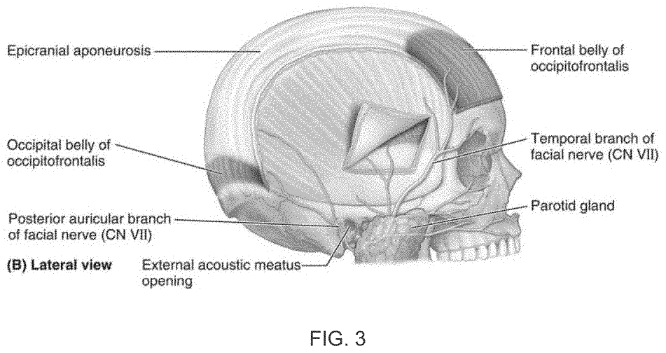

FIG. 3 is a drawing of the anatomy of the temporal area/temporal bone anatomy and the cranial spaces.

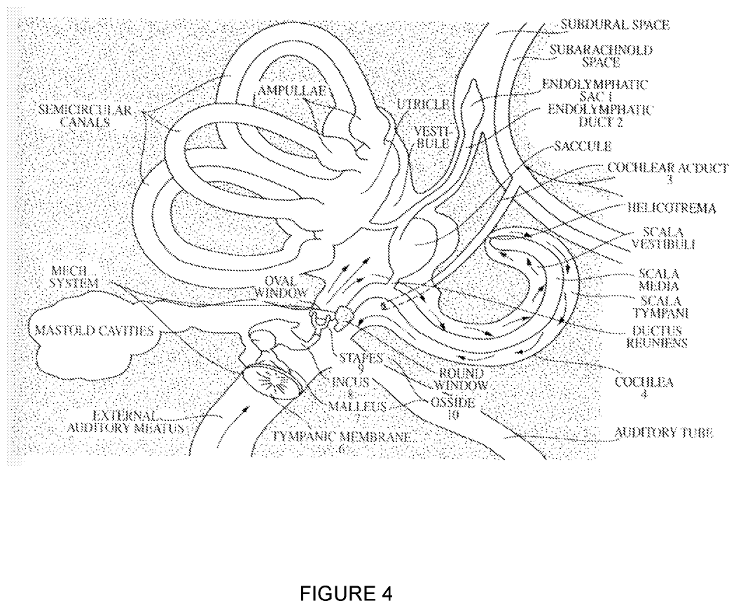

FIG. 4 is a drawing of the anatomy of the ear and the cranial spaces showing the communication between the cranial spaces and the oval window of the inner ear. The drawing generally depicts the anatomy of the ear and surrounding cranial spaces. The sensor 220 reaches within the external auditory meatus and targets the oval window. in the healthy ear, the pressure within the oval window is equivalent to the ICP). The oval window is in isostasy with CSF pressure, it is ‘intracranial’ and not affected by otitis, middle ear fluid, the tympanic membrane etc.

While the invention may be susceptible to various modifications and alternative forms, specific embodiments have been shown by way of example in the drawings and will be described in detail herein. However, it should be understood that the invention is not intended to be limited to the particular forms disclosed. Rather, the invention is to cover all modifications, equivalents and alternatives falling within the spirit and scope of the invention as defined by the following appended claims.

What is claimed is:

1. An intracranial pressure (ICP) measurement system comprising:
a sensor, the sensor further comprising:
a transmitter for transmitting an electromagnetic signal through a skull bone to an intracranial area; and
a receiver for receiving a reflected electromagnetic signal;
a central processor unit to receive the reflected electromagnetic signal from the receiver and reconstruct the reflected electromagnetic signal into an ICP wave; and
a processor to analyze the ICP wave and determine an ICP pressure.

2. The ICP measurement system of claim 1, wherein the sensor comprises an adhesive layer for attaching to a patient temple area.

3. The ICP measurement system of claim 1, wherein the sensor comprises an extended end for fitting in a patient ear canal.

4. The ICP measurement system of claim 1, wherein the transmitter and the receiver are a single antenna.

5. A method for measuring an ICP, the method comprising:
placing a sensor in proximity to a patient head structure, the sensor further comprising a transmitter and a receiver;
transmitting an electromagnetic signal from the sensor through a skull bone to an intracranial area;
receiving a reflected electromagnetic signal to the sensor;
transmitting the reflected electromagnetic signal from the sensor to a central processing unit;
reconstructing the reflected electromagnetic signal to obtain an ICP wave; and
analyzing the ICP wave to determine an ICP.

6. The method of claim 5, wherein the patient head structure comprises a temple area.

7. The method of claim 5, wherein the patient head structure comprises an ear canal.
